(19) United States
(12) Patent Application Publication
(10) Pub. No.: US 2012/0245493 A1
(43) Pub. Date: Sep. 27, 2012

(54) ULTRASOUND NEUROMODULATION TREATMENT OF ADDICTION

(76) Inventor: David J. Mishelevich, Playa del Rey, CA (US)

(21) Appl. No.: 13/425,436

(22) Filed: Mar. 21, 2012

Related U.S. Application Data
(60) Provisional application No. 61/454,746, filed on Mar. 21, 2011.

(51) Int. Cl. A61N 7/00

(52) U.S. Cl. 601/2

(57) ABSTRACT

Disclosed are methods and systems and methods for non-invasive neuromodulation using ultrasound to treat addiction. The neuromodulation can produce acute or long-term effects. The latter occur through Long-Term Depression (LTD) and Long-Term Potentiation (LTP) via training. Included is control of direction of the energy emission, intensity, frequency, pulse duration, and phase/intensity relationships to targeting and accomplishing up regulation and/or down regulation.

### CROSS REFERENCE TO RELATED APPLICATIONS

This patent application claims priority to Provisional Patent Application No. 61/454,746, filed Mar. 21, 2011, entitled “ULTRASOUND NEUROMODULATION TREATMENT OF ADDICTION.”

This patent application may be related to one or more of the following patents and pending patent applications (US and PCT applications), each of which is herein incorporated by reference in its entirety: U.S. patent application Ser. No. 12/940,052, titled “NEUROMODULATION OF DEEP-BRAIN TARGETS USING FOCUSED ULTRASOUND,” filed on Nov. 5, 2010; U.S. patent application Ser. No. 12/958,411, titled “MULTI-MODALITY NEUROMODULATION OF BRAIN TARGETS,” filed on Dec. 2, 2010; U.S. patent application Ser. No. 13/007,626, titled “PATIENT FEEDBACK FOR CONTROL OF ULTRASOUND DEEP-BRAIN NEUROMODULATION,” filed on Jan. 15, 2011; U.S. patent application Ser. No. 13/200,903, titled “SHAPED AND STEERED ULTRASOUND FOR DEEP-BRAIN NEUROMODULATION,” filed on Jan. 15, 2011; U.S. patent application Ser. No. 13/020,016, titled “ULTRASOUND NEUROMODULATION OF THE SPHENOPALATINE GANGLION,” filed on Feb. 3, 2011; U.S. patent application Ser. No. 13/021,785, titled “ULTRASOUND NEUROMODULATION OF THE OCCIPUT,” filed on Feb. 7, 2011; U.S. patent application Ser. No. 13/031,192, titled “ULTRASOUND NEUROMODULATION OF THE RETICULAR ACTIVATING SYSTEM,” filed on Feb. 19, 2011; U.S. patent application Ser. No. 13/035,962, titled “ORGASMATRON VIA DEEP-BRAIN NEUROMODULATION,” filed on Feb. 26, 2011; U.S. patent application Ser. No. 13/098,473, titled “ULTRASOUND MACRO-PULSE AND MICRO-PULSE SHAPES FOR NEUROMODULATION,” filed on May 1, 2011; U.S. patent application Ser. No. 13/252,054, titled “ULTRASOUND-INTERSECTING BEAMS FOR DEEP-BRAIN NEUROMODULATION,” filed on Oct. 3, 2011; U.S. patent application Ser. No. 13/360,600, titled “PATTERNED CONTROL OF ULTRASOUND FOR NEUROMODULATION,” filed on Jan. 27, 2012; U.S. patent application Ser. No. 13/405,337, titled “ULTRASOUND NEUROMODULATION FOR STROKE MITIGATION AND REHABILITATION,” filed on Feb. 26, 2012; U.S. patent application Ser. No. 13/411,641, titled “ULTRASOUND NEUROMODULATION TREATMENT OF PAIN,” filed on Mar. 5, 2012; and U.S. patent application Ser. No. 13/413,659, titled “ULTRASOUND NEUROMODULATION TREATMENT OF TINNITUS,” filed on Mar. 7, 2012.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation including one or more ultrasound sources for neuromodulation of target deep brain regions to up-regulate or down-regulate neural activity.

### BACKGROUND OF THE INVENTION

It has been demonstrated that focused ultrasound directed at neural structures can stimulate those structures. If neural activity is increased or excited, the neural structure is said to be up regulated; if neural activated is decreased or inhibited, the neural structure is said to be down regulated. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested. The above approach is incorporated in a patent application submitted by Tyler (Tyler, William, James P., PCT/US2009/050560, WO 2010/009141, published 2011-01-21).

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. Bystritsky in U.S. Pat. No. 7,283,861 notes differences in FUP phase, frequency, and amplitude produce different neural effects. This patent also notes low frequencies (defined as below 300 Hz) inhibit neural circuits and high frequencies (defined as being in the range of 500 Hz to 5 MHz) are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

Deisseroth and Schneider (U.S. patent application Ser. No. 12/263,026 published as US 2009/0112133 A1, Apr. 30, 2009) describe an alternative approach in which modifications of neural transmission patterns between neural structures and/or regions are described using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24(2):275-83 and Clement G T, Hynynen K (2002) A non-invasive method for focusing ultrasound through the human skull. Phys Med Biol 47: 1219-1236.). Ultrasound can be focused to 0.5 to 2 mm as TMS to 1 cm at best.

Because of the utility of ultrasound in the neuromodulation of deep-brain structures, it would be both logical and desirable to apply it to the treatment of addiction.

### SUMMARY OF THE INVENTION

It is the purpose of this invention to provide methods and systems for non-invasive neuromodulation using transcranial ultrasound neuromodulation to treat addiction. Such neuromodulation can produce acute effects or Long-Term Potentiation (LTP) or Long-Term Depression (LTD). Included is control of direction of the energy emission, intensity, frequency, pulse duration, and phase/intensity relationships to targeting and accomplishing up-regulation and/or down-regulation. Use of ancillary monitoring or imaging to provide feedback is optional. In embodiments where concurrent imaging is performed, the device of the invention is constructed of non-ferrous material.

Multiple targets can be neuromodulated singly or in groups to treat addiction. To accomplish the treatment, in some cases the neural targets will be up regulated and in some cases down regulated, depending on the given neural target. Targets have been identified by such methods as PET imaging, fMRI imaging, and clinical response to Transcranial Magnetic Stimulation (TMS). Currently, the Orbito-Frontal Cortex (OFC)(Wang Z., Faith, M., Patterson, F., Tang, K., Kerrin, K., Wileyto, E. P., Detre, J. A., and C. Lerman, “Neural substrates of abstinence-induced cigarette cravings in chronic smokers,” J Neurosci. 2007 Dec. 19; 27(51):14035-40), the Dorsal Anterior Cingulate Gyms (DACG)(Goldstein, Rita Z., Alia-Kleina, Nelly, Tomasia, D., Honorio Carrillo, J., Maloneya, T., Patricia A. Woicika, Wanga, R., Telang, F., and Nora D. Volkow, “Anterior cingulate cortex hypoactivations to an emotionally salient task in cocaine addiction,” PNAS, 106(23): 9453-9458, Jun. 9, 2009, www.pnas.org_cgi_doi_{—}10.1073_pnas.0900491106), the Insula (Naqvi, N. H., Rudrauf, D., Damasio, Hanna, and A. Bechara, “Damage to the Insula Disrupts Addiction to Cigarette Smoking” (abstract). Science 315 (5811): 531-4), January 2007), Nucleus Accumbens (Di Chiara, G., Bassareo, V., Fenu, S., De Luca, M. A., Spina, L., Cadoni, C., Acquas, E., Carboni, E., Valentini, V., and D. Lecca, “Dopamine and drug addiction: the nucleus accumbens shell connection,” Neuropharmacology. 2004; 47 Suppl 1:227-410, and the Globus Pallidus (Miller, J. M., Vorel, S. R., Tranguch, A. J., Kenny, E. T., Mazzoni, P., van Gorp, W. G., and H. D. Kleber, “Anhedonia After a Selective Bilateral Lesion of the Globus Pallidus,” Am J Psychiatry 163:786-788, May 2006, doi: 10.1176/appi.ajp.163.5.786) would all be down regulated. In some cases neuromodulation will be bilateral and in others unilateral. The specific targets and/or whether the given target is up regulated or down regulated, can depend on the individual patient and relationships of up regulation and down regulation among targets, and the patterns of stimulation applied to the targets.

The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target.

### BRIEF DESCRIPTION OF THE DRAWINGS

FIG. 1 shows ultrasonic-transducer targeting of the Orbito-Frontal Cortex (OFC), the Dorsal Anterior Cingulate Gyms (DACG) and the Insula.

FIG. 2 shows a block diagram of the control circuit.

### DETAILED DESCRIPTION OF THE INVENTION

It is the purpose of this invention to provide methods and systems and methods for neuromodulation of deep-brain targets using ultrasound to treat addiction. Such neuromodulation systems can produce applicable acute or long-term effects. The latter occur through Long-Term Depression (LTD) or Long-Term Potentiation (LTP) via training. Included is control of direction of the energy emission, intensity, frequency, pulse duration, and phase/intensity relationships to targeting and accomplishing up-regulation and/or down-regulation.

The stimulation frequency for inhibition is approximately 400 Hz or lower (depending on condition and patient). In one embodiment, the modulation frequency of lower than approximately 400 Hz is divided into pulses 0.1 to 20 msec. repeated at frequencies of 2 Hz or lower for down regulation. The stimulation frequency for excitation is in the range of 600 Hz to 6 MHz. In one embodiment, the modulation frequency of higher than approximately 600 Hz. is divided into pulses 0.1 to 20 msec. repeated at frequencies higher than 2 Hz for up regulation. In this invention, the ultrasound acoustic frequency is in range of 0.3 MHz to 0.8 MHz with power generally applied less than 60 mW/cm² but also at higher target- or patient-specific levels at which no tissue damage is caused. In some embodiments of the invention, the acoustic frequency is gated at the lower rate to impact the neuronal structures as desired (e.g., say 400 Hz for inhibition (down-regulation) or 1 kHz for excitation (up-regulation). Ultrasound therapy can be combined with therapy using other devices (e.g., Transcranial Magnetic Stimulation (TMS)).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space.

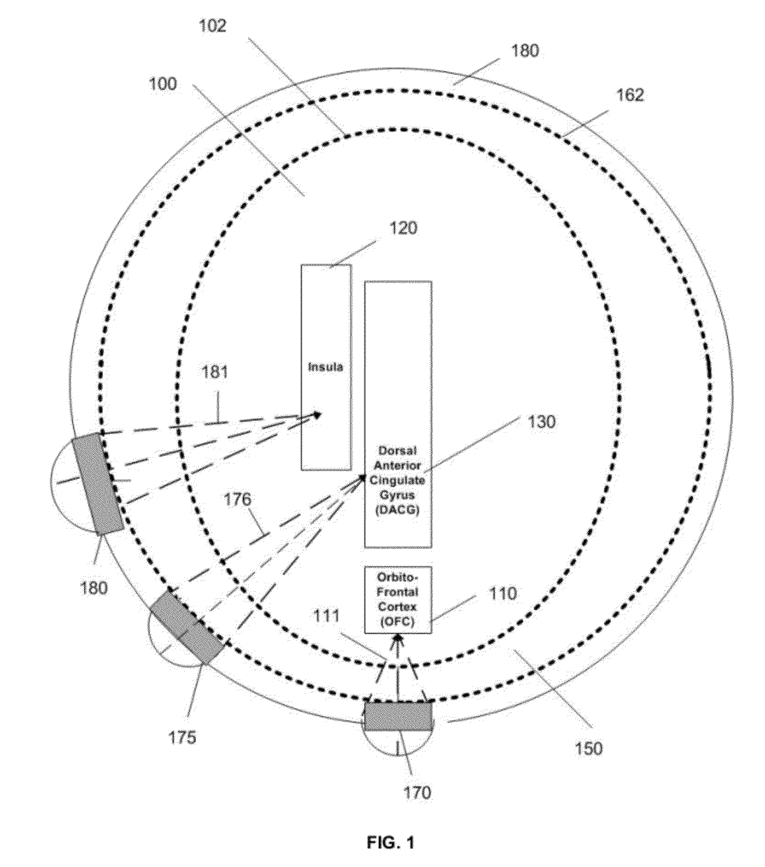

FIG. 1 shows a set of ultrasound transducers targeting to treat addiction. The head 100 contains the three targets, Orbito-Frontal Cortex (OFC) 110, Insula 120, and Dorsal Anterior Cingulate Gyms (DACG) 130. These targets are hit by ultrasound from transducers 170, 175, and 180 fixed to track 160. Ultrasound transducer 170 with its beam 111 is shown targeting the OFC 110, transducer 175 with its beam 176 is shown targeting the DACG 130, and transducer 180 with its beam 181 is shown targeting the Insula 120. For ultrasound to be effectively transmitted to and through the skull and to brain targets, coupling must be put into place. Ultrasound transmission (for example Dermasol from California Medical Innovations) medium 150 is interposed with one mechanical interface to the frame 180 and ultrasound transducers 170, 175, 180 (completed by a layer of ultrasound transmission gel 162) and the other mechanical interface to the head 100 (completed by a layer of ultrasound transmission gel 102). In another embodiment the ultrasound transmission gel is only placed at the particular places where the ultrasonic beams from the transducers are located rather than around the entire frame and entire head.

Transducer array assemblies of this type may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Keramos-Etalon in the U.S. is another custom-transducer supplier. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

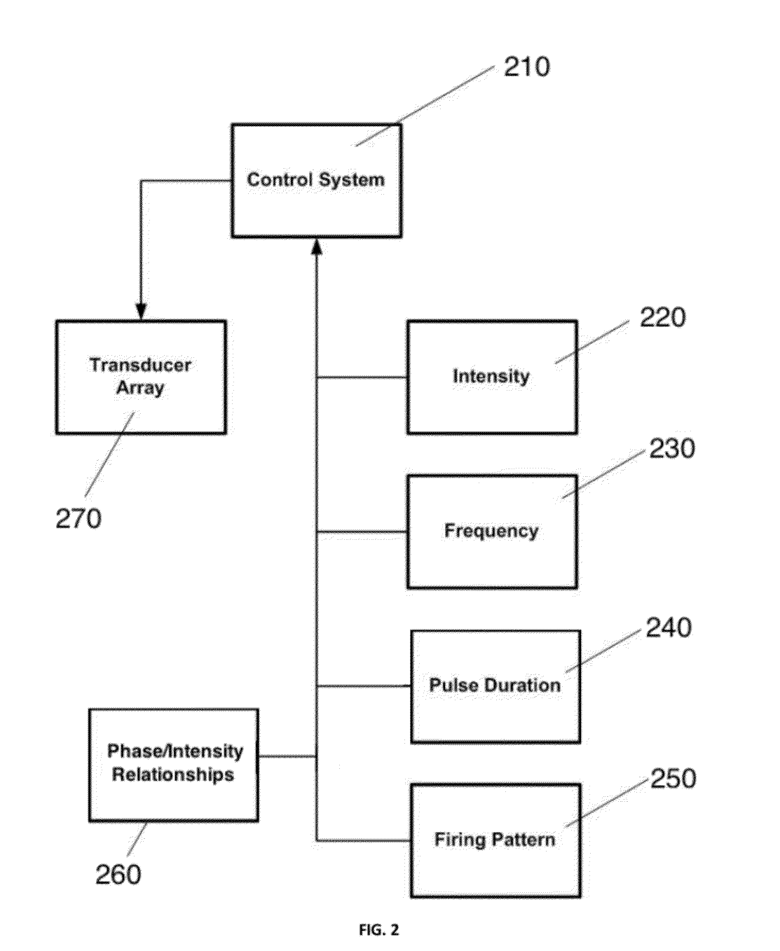

FIG. 2 shows an embodiment of a control circuit. The positioning and emission characteristics of transducer array 270 are controlled by control system 210 with control input with neuromodulation characteristics determined by settings of intensity 220, frequency 230, pulse duration 240, firing pattern 250, and phase/intensity relationships 260 for beam steering and focusing on neural targets.

In another embodiment, a feedback mechanism is applied such as functional Magnetic Resonance Imaging (fMRI), Positive Emission Tomography (PET) imaging, video-electroencephalogram (V-EEG), acoustic monitoring, thermal monitoring, and patient feedback.

In still other embodiments, other energy sources are used in combination with or substituted for ultrasound transducers that are selected from the group consisting of Transcranial Magnetic Stimulation (TMS), deep-brain stimulation (DBS), transcranial Direct Current Stimulation (tDCS), optogenetics application, radiosurgery, Radio-Frequency (RF) therapy, and medications.

The invention allows stimulation adjustments in variables such as, but not limited to, intensity, firing pattern, frequency, pulse duration, phase/intensity relationships, dynamic sweeps, and position.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of deep-brain neuromodulation using ultrasound stimulation, the method comprising:
aiming an plurality of ultrasound transducer at one or a plurality of addiction-related neural targets, and
applying pulsed power to the ultrasound transducer via a control circuit,
whereby addiction is alleviated.

2. The method of claim 1, further comprising aiming an ultrasound transducer neuromodulating addiction-related neural targets in a manner selected from the group of up-regulation, down-regulation.

3. The method of claim 1, wherein the effect is chosen from the group consisting of acute, Long-Term Potentiation, and Long-Term Depression.

4. The method of claim 1, wherein one or a plurality of targets are selected from the group consisting of Orbito-Frontal Cortex, Dorsal Anterior Cingulate Gyms, Insula, Nucleus Accumbens, and Globus Pallidus.

5. The method of claim 1, wherein the acoustic ultrasound frequency is in the range of 0.3 MHz to 0.8 MHz.

6. The method of claim 1, where in the power applied is less than 60 mW/cm².

7. The method of claim 1, wherein the power applied is greater than 60 mW/cm² but less than that causing tissue damage.

8. The method of claim 1, wherein a stimulation frequency less than approximately 400 Hz or lower is applied for inhibition of neural activity.

9. The method of claim 8 wherein modulation frequency of lower than approximately 400 Hz is divided into pulses 0.1 to 20 msec. repeated at frequencies of 2 Hz or lower for down regulation.

10. The method of claim 1, wherein the stimulation frequency for excitation is in the range of 600 Hz to 6 MHz.

11. The method of claim 10 wherein modulation frequency of approximately 600 Hz or higher is divided into pulses 0.1 to 20 msec. repeated at frequencies higher than 2 Hz for up regulation.

12. The method of claim 1, wherein the focus area of the pulsed ultrasound is 0.5 to 50 mm in diameter.

13. The method of claim 1, wherein the focus area of the pulsed ultrasound is 50 to 150 mm in diameter.

14. The method of claim 1, wherein the number of ultrasound transducers is between 1 and 10.

15. The method of claim 1, wherein mechanical perturbations are applied radially or axially to move the ultrasound transducers.

16. The method of claim 1, wherein a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, patient.

17. The method of claim 1, wherein ultrasound therapy is combined with or replaced by one or more therapies selected from the group consisting of Transcranial Magnetic Stimulation (TMS), deep-brain stimulation (DBS), transcranial Direct Current Stimulation (tDCS), application of optogenetics, radiosurgery, Radio-Frequency (RF) therapy, and medications
